(19) United States
(12) Patent Application Publication
(10) Pub. No.: US 2022/0313686 A1
(43) Pub. Date: Oct. 6, 2022

(54) PHARMACEUTICAL COMPOSITIONS COMPRISING A COMBINATION OF OPIOID ANTAGONISTS

(71) Applicant: YISSUM RESEARCH DEVELOPMENT COMPANY OF THE HEBREW UNIVERSITY OF JERUSALEM LTD., Jerusalem (IL)

(72) Inventors: Yechezkel BARENHOLZ, Jerusalem (IL); Ahuva CERN, Modiin (IL)

(21) Appl. No.: 17/634,437

(22) PCT Filed: Aug. 12, 2020
(86) PCT No.: PCT/IL2020/050884

§ 371 Date: Feb. 10, 2022

Related U.S. Application Data
(60) Provisional application No. 62/885,569, filed on Aug. 12, 2019.

(51) Int. Cl. A61K 31/485; A61K 31/5377; A61K 9/127

(52) U.S. Cl. CPC A61K 31/485; A61K 31/5377; A61K 9/127

(57) ABSTRACT

The present disclosure provides pharmaceutical compositions comprising a first opioid antagonist and a second opioid antagonist, wherein the first opioid antagonist has a half-life in plasma that is of shorter duration than the half-life of the second opioid antagonist in the plasma and wherein the second opioid antagonist is encapsulated within liposomes. The composition may be embedded or entrapped within a water insoluble, water absorbable polymeric matrix, to form a composite water. The pharmaceutical composition or the composite material can be used in a method of counteracting opioid overdose in a subject by administering the same, preferably by intramuscular injection, to the subject.

### TECHNOLOGICAL FIELD

The present invention concerns drug delivery and specifically for the delivery of opioid antagonists either alone or in combination with other active ingredients such as respiratory stimulators.

### BACKGROUND

Synthetic opioids (e.g. carfentanyl) can be weaponized to create a surge in opioid overdoses that can overwhelm available emergency resources and supplies. Current treatment options often require multiple doses to be effective; in a large-scale Attack repeat doses of current countermeasures may not be feasible. Consequently, it is desirable to develop fast-onset, long-acting opioid antagonist(s) effective against weaponized high potency opioids. Opioid formulations could be efficiently deployed in a variety of scenarios including public health situations or terrorist mass-casualty scenarios.

### GENERAL DESCRIPTION

The present disclosure provides a pharmaceutical composition comprising as an active ingredient, at least two active components including a first opioid antagonist and a second opioid antagonist, wherein the first opioid antagonist has a half-life in plasma that is shorter than the half-life of the second opioid antagonist in the plasma and wherein the second opioid antagonist is encapsulated within liposomes.

Also disclosed herein is a composite material comprising a water insoluble, water absorbable polymeric matrix, and embedded or entrapped within the matrix, as an active ingredient at least two active components including a first opioid antagonist and a second opioid antagonist, wherein the first opioid antagonist has a half-life in plasma that is shorter than the half-life of the second opioid antagonist in the plasma and wherein the second opioid antagonist is encapsulated within liposomes.

The composition and/or composite material disclosed herein could be efficiently deployed in a variety of scenarios including public health situations or terrorist mass-casualty scenarios. The composition and/or composite material may also provide an effective treatment for situations in which adulterated pills infiltrate a community.

A specific, yet non limiting example for a first opioid antagonist and a second opioid antagonist includes, respectively, Naloxone (also known as N-allylnoroxymorphone or as 17-allyl-4,5α-epoxy-3,14-dihydroxymorphinan-6-one or (4R,4aS,7aR,12bS)-4a,9-dihydroxy-3-prop-2-enyl-2,4,5,6,7a,13-hexahydro-1H-4,12-methanobenzofuro[3,2-e]isoquinolin-7-one) and Naltrexone (also known as N-Cyclopropylmethylnoroxymorphone or (4R,4a,7aR,12bS)-3-(cyclopropylmethyl)-4a,9-dihydroxy-2,4,5,6,7a,13-hexahydro-1H-4,12-methanobenzofuro[3,2-e]isoquinolin-7-one).

The compositions of the present invention can include other active compounds/components including respiratory stimulants such as but not limited to doxapram (1-ethyl-4-(2-morpholin-4-ylethyl)-3,3-diphenylpyrrolidin-2-one), as further discussed below.

The present disclosure also provides the composition or composite matter for use or a method of providing a prolonged counteraction against opioid overdose, the method comprises administration to a subject suffering from opioid overdose an amount of a pharmaceutical composition comprising as active ingredients at least a first opioid antagonist and a second opioid antagonist, wherein the first opioid antagonist has a half-life in plasma that is shorter than the half-life of the second opioid antagonist in the plasma and wherein the second opioid antagonist is encapsulated within liposomes; or composite material comprising a water insoluble, water absorbable polymeric matrix, having embedded or entrapped within the matrix a first opioid antagonist and a second opioid antagonist, wherein the first opioid antagonist has a half-life in plasma that is shorter than the half-life of the second opioid antagonist in the plasma and wherein the second opioid antagonist is encapsulated within liposomes.

In some examples, the method comprises intramuscular administration of the pharmaceutical composition or of the composite material.

### DETAILED DESCRIPTION OF EMBODIMENTS

The present disclosure is aimed at providing a liposomes comprising composition or composite material comprising the liposomal composition, the latter comprising a dual opioid counteracting effect that may suitable for mass casualty scenarios, such as during chemical warfare.

In addition, the liposomal composition or composite material comprising the same, both disclosed herein can be of advantage treating opioid overdose where the liposomal formulation can be intramuscularly injected by the individual in need of said treatment, without the aid of a physician or other medical care provider.

Specifically, and in accordance with a first of its aspects, the present disclosure provides a pharmaceutical composition comprising as active ingredients, a first opioid antagonist and a second opioid antagonist, wherein the first opioid antagonist has a half-life in plasma that is shorter than the half-life of the second opioid antagonist in the plasma and wherein the second opioid antagonist is at least encapsulated within liposomes.

Also disclosed herein is a composite material comprising a water insoluble, water absorbable polymeric matrix embedding or entrapping at least a portion of the first opioid antagonist and the second opioid antagonist and any additional active ingredients.

In the context of the present disclosure, the active ingredients, namely, the first opioid antagonist, the second opioid antagonist (within liposomes as well as in free form) and any additional active ingredients are collectively referred to by the term “pharmaceutical composition”, and this pharmaceutical composition being embedded within the polymeric matrix is referred to herein by the term “composite material”.

An opioid antagonist is a compound, typically a low molecular weight compound that blocks opioids by attaching to the opioid receptors without activating them, namely, without causing an opioid effect.

In some examples, the first opioid antagonist is one typically employed for treating acute opioid overdose, where there is need for an immediate blockage of the opioid receptors. Accordingly, in some examples, the first opioid antagonist is one that acts within a few minutes from administration and lasts for a short period of time, because of a rapid metabolism.

In some examples, the first opioid antagonist is Naloxone (N-Allylnoroxymorphone; 17-allyl-4,5α-epoxy-3,14-dihydroxymorphinan-6-one HCl), a μ-opioid receptor antagonist also known by the brand name Narcan. The half-life in the plasma of Naloxone is about 1-1.5 hour from administration.

The second opioid antagonist is one having a longer half-life in plasma that is longer than that of the first opioid antagonist.

When referring to a shorter or longer half-life between the two opioid antagonists it is to be understood that the first opioid antagonist has a half-life in the plasma that is at least 20%, at least 30%, at least 40%, at least 50% or even at least 75% shorter than the half-life of the second opioid antagonist.

In some examples, the second opioid antagonist is Naltrexone [N-Cyclopropylmethylnoroxymorphone; N-Cyclopropylmethyl-14-hydroxydihydro-morphinone; 17-(cyclopropylmethyl)-4,5α-epoxy-3,14-dihydroxymorphinan-6-one], a μ-opioid receptor antagonist, having a reported half-life of approximately 3½ hours [Yuen K H et al., “Comparative bioavailability study of a generic naltrexone tablet preparation” Drug Dev Ind Pharm 25:353-356, (1999)] with a 5-fold higher affinity for the receptor compared with naloxone [Cassel J A et al., “[3 H]Alvimopan binding to the m opioid receptor: comparative binding kinetics of opioid antagonists”. Eur J Pharmacol 520:29-36, (2005)].

An alternative to Naltrexone may be Samidorphan (SAM, 3-carboxamido-4-hydroxynaltrexone), a more recently developed, novel opioid-system modulator, primarily functions as an MOR antagonist in vivo [Chaudhary A, Khan M F, Dhillon S S, et al. “A Review of Samidorphan: A Novel Opioid Antagonist”. Cureus 11(7): e5139. (Jul. 15, 2019) doi:10.7759/cureus.5139]. It is structurally related to naltrexone, yet, compared to naltrexone, SAM has a five-fold greater affinity at mu-opioid receptor and much greater bioavailability when administered orally. In vitro, it has a high affinity at μ-receptor, κ-receptor and δ-receptor; and it acts as an antagonist at μ-receptor and a partial agonist at κ and δ-receptor [Chaudhary A. ibid. 2019].

Thus, while Naloxone acts within minutes and lasts for about an hour, Naltrexone provides a long-lasting effect not only due to its greater half-life in the plasma but also due to its encapsulation within liposomes which prolong its circulation time. In addition, the metabolite of naltrexone, 60-naltrexol is also an active antagonist. So, the effects of naltrexone arise from both the parent drug and its major metabolite and last about a day after its release from the liposome.

Additional combinations of opioids antagonists may include nalbuphine, butorphanol, pentazocine, diprenorphine and dihydroetorphine as well as opioid alkaloids and opioid peptides.

The pharmaceutical composition comprises, as disclosed hereinabove, the second opioid antagonist, within liposomes, and the first opioid antagonist being in free form.

In some examples, at least a portion of the first opioid antagonist is also within liposomes.

In some examples, at least a portion of the second opioid antagonist is within the same liposome as the first opioid antagonist; i.e. the liposomes encapsulate both the first opioid antagonist and the second opioid antagonist.

In some examples, the pharmaceutical composition comprises at least one additional active ingredient.

In some examples, the additional active ingredient is also embedded in the polymeric matrix in free form.

In some examples, the additional active ingredient is a respiratory stimulant. A non-limiting example of a respiratory stimulant is doxapram hydrochloride (1-ethyl-4-(2-morpholin-4-ylethyl)-3,3-diphenyl-pyrrolidin-2-one, also marketed under the brand names Dopram, Stimulex or Respiram) and Zacopride (4-amino-5-chloro-2-methoxy-N-(quinuclidin-3-yl)benzamide).

A non-limiting example of a combination of the first opioid antagonist, e.g. Naloxone and the second opioid antagonist, e.g. Naltrexone may include the following injectable doses:

| | Max injectable | D/L molar ratio required for max |
|---|---|---|
| Opioid | dose (mg/day) | injectable dose* |
| Naltrexone | 50 mg tablet | 0.375 |
| | equivalent to <20 mg | |
| | injection | |
| Naloxone | ≤10 mg | 0.1875 |

*Assuming lipid concentration of 40 mM and injection vol. of 4 ml

The combination of the first opioid antagonist in free form and the second opioid antagonist being at least within liposomes (i.e. some may be external to the liposomes) and optionally additional active ingredients, embedded or entrapped within a polymeric matrix, specifically, hydrogel, to form the composite material disclosed herein, can improve duration of action, e.g. to provide a long lasting, e.g. a 48-96 hour duration of action of the opioids and additional active ingredients.

The polymeric matrix in which the composition is entrapped or embedded comprises at least one water insoluble, water absorbent/absorbable polymer. Such polymers are known to form in an aqueous environment a hydrogel.

As used herein, the term “matrix” denotes any network or network-like scaffold that may be formed from a fully cross-linked or partially cross-linked or non cross-linked polymer and is capable of confining at least a portion of the pharmaceutical composition, i.e. the free and the liposomal opioids. Thus, it is to be understood that hereinabove and below, when referring to a polymer, it also encompasses more than one polymer forming the matrix.

The cross-linked polymer forms a water insoluble (water immiscible) matrix. The term “water insoluble” is used to denote than upon contact with water or a water containing fluid the polymeric matrix does not dissolve or disintegrates.

Further, in the context of the present disclosure, the polymeric matrix is biocompatible, i.e. is inert to body tissue, such that upon administration to a body, it will not be toxic, injurious, physiologically reactive or cause any immunological rejection of the composition of matter.

The polymeric matrix is also a water absorbing matrix and in the context of the present disclosure is absorbed or can absorb water. As used herein, the term “water absorbing” or “water absorbed” is used to denote that the polymer, once formed into a matrix is capable of absorbing water in an amount that is at least 4 times, at times 10-50 times and even more of the polymer's or polymers' own weight thereby forming a gel or a hydrogel.

The polymer(s) forming the matrix can be a naturally occurring polymer or a synthetic or semi-synthetic polymer.

In some examples, the matrix forms a hydrogel that is a thermal responsive cross-linked hydrogel.

In some examples, the polymeric matrix comprises a fully cross-linked water absorbing polymer, a partially cross-linked water absorbing polymer in non-cross linked polymers. In some examples, a cross-linked polymer (fully or partially) is used.

Water absorbing cross-linkable polymers generally fall into three classes, namely, starch graft copolymers, cross-linked carboxymethylcellulose derivatives, and modified hydrophilic polyacrylates. Examples of absorbent polymers are hydrolyzed starch-acrylonitrile graft copolymer; a neutralized starch-acrylic acid graft copolymer, a saponified acrylic acid ester-vinyl acetate copolymer, a hydrolyzed acrylonitrile copolymer or acrylamide copolymer, a modified cross-linked polyvinyl alcohol, a neutralized self-cross-linking polyacrylic acid, a cross-linked polyacrylate salt, carboxylated cellulose, and a neutralized cross-linked isobutylene-maleic anhydride copolymer.

In some examples of the composite material of the present disclosure, the polymeric matrix is soaked with water thereby forming a hydrogel.

In some examples, the matrix is a “hydrogel”. The term “hydrogel” as used herein has the meaning acceptable in the art. Generally, the term refers to a class of highly hydratable polymer materials typically composed of hydrophilic polymer chains, which may be naturally occurring, synthetic or semi synthetic and crossed linked (fully or partially).

In some examples, the polymeric matrix, e.g. the hydrogel, is an injectable matrix.

Injectable hydrogels have been widely investigated for various proposes due to their perfect biocompatibility, biodegradability, and similarity to the native ECM. Natural biomaterials, such as chitosan and hyaluronic acid, alginic acid, PLGA-PEG-PLGA Triblock Copolymer can generate a three-dimensional (3D) hydrogels entrapping nano to micro particles and contribute to higher bio-adhesively and site-specificity effect and may help to control drug administration in the desire site as further discussed below.

In one example embodiment, the matrix is a “hydrogeF. The term “hydrogeF” as used herein has the meaning acceptable in the art. Generally, the term refers to a class of highly hydratable polymer materials typically composed of hydrophilic polymer chains, which may be naturally occurring, synthetic or semi synthetic and crossed linked (fully or partially).

Synthetic polymers that are known to form hydrogels include, without being limited thereto, poly(ethylene oxide) (PEO), poly(vinyl alcohol) (PVA), poly(acrylic acid) (PAA), poly(propylene furmarate-co-ethylene glycol) (P(PF-co-EG)), and polypeptides. Representative naturally occurring, hydrogel forming polymers include, without being limited thereto, agarose, alginate, chitosan, collagen, fibrin, gelatin, and hyaluronic acid (HA). A subset of these hydrogels includes PEO, PVA, P(PF-co-EG), alginate, hyaluronate (HA), chitosan, and collagen.

In some examples of the present disclosure, the polymeric matrix comprises alginate, such as, and at times preferably, low viscosity (LV) alginate (molecular weight of the polycarbohydrate ˜100,000), or very low viscosity (VLV) alginate (molecular weight of the polycarbohydrate ˜30,000). The alginate may be cross linked by Ca ions to from Ca-alginate cross-linked hydrogel. The cross-linked alginate is a water absorbing polymer, forming in the presence of water a hydrogel.

In some embodiments, the matrix comprises partially or fully cross-linked polymer(s).

In yet some other embodiments, the matrix comprises at least one cross-linked polysaccharide.

In one example, the matrix is a Hyaluronate Hyaluronsan HA-AM hydrogel. The Hyaluronate Hyaluronsan HA-AM hydrogel is a negatively charged hydrogel (MW molecular weight: 600,000 to 1,200,000 and intrinsic viscosity: 11.8-19.5 dl/g) formed from hyaluronic acid an calcium ions.

In one other example, the matrix comprises chitosan cross-linked with oxalic acid to form a positively charged hydrogel.

In one further example, the hydrogel comprises alginate that is cross-linked by Ca ions to from Ca-alginate cross-linked hydrogel.

In one further example, the hydrogel comprises PLGA-PEG-PLGA triblock copolymer, the synthesis procedure of which was previously described [Steinman, N. Y., Haim-Zada, M., Goldstein, I. A., Goldberg, A. H., Haber, T., Berlin, J. M. and Domb, A. J. (2019), Effect of PLGA block molecular weight on gelling temperature of PLGA-PEG-PLGA thermoresponsive copolymers. J. Polym. Sci. Part A: Polym. Chem., 57: 35-39. doi:10.1002/pola.29275].

To obtain the composite material, the pharmaceutical composition comprising the liposome and the free opioid can be added, typically slowly and under stirring conditions, to the polymer solution, after which the cross-linking takes place, e.g. by the addition of the cross-linkers, such as, and without being limited thereto, the calcium ions or oxalic acid mentioned above.

In one embodiment, the polymer forming the matrix is biodegradable. The term “biodegradable” refers to the degradation of the polymer by one or more of hydrolysis, enzymatic cleavage, and dissolution. In this connection, when the matrix is a hydrogel comprising synthetic polymer, degradation typically is based on hydrolysis of ester linkages, although not exclusively. As hydrolysis typically occurs at a constant rate in vivo and in vitro, the degradation rate of hydrolytically labile gels (e.g. PEG-PLA copolymer) can be manipulated by the composition of the matrix. Synthetic linkages have also been introduced into PEO to render it susceptible to enzymatic degradation. The rate of enzymatic degradation typically depends both on the number of cleavage sites in the polymer and the amounts of available enzymes in the environment. Ionic cross-linked alginate and chitosan normally undergoes de-crosslinking and dissolution but can also undergo controlled hydrolysis after partial oxidization. The rate of dissolution of ionic crosslinked alginate and chitosan depends on the ionic environment in which the matrix is placed. As will be illustrated below by one embodiment it is possible to use cross-linked polymer and control the rate of degradation by addition at a desired time and a desired amount of a de-crosslinker.

Specific examples of control of the cross-linking and de-crosslinking may include cross-linking the cationic chitosan with the di-carboxylic acid oxalate (OA) and de-cross-linking by the divalent cation calcium; and cross-linking the anionic alginate with the divalent cation calcium and de-crosslinking by either di-carboxylic acid such as oxalate (OA) or by chelating agents such as EDTA. Thus, at times, the composition of matter may be subjected to de cross linking.

When referring to water absorbing non-cross-linked polymers. For example, the gel can be a PEG based gel, such as the non-limiting example of PEG-PLGA gel disclosed herein.

Being soaked with an aqueous medium, the composite material, namely, the polymeric matrix holding the liposomes, is in liquid or semi-liquid form.

The liposomes and specifically the composite material is used for local delivery of the opioids and additional active ingredients, preferably, for local controlled delivery.

The polymeric matrix may be present in the composite material in the form of individual particles, e.g. beads, each particle embedding liposomes and all being within a medium carrying the free opioid(s), or the pharmaceutical composition is embedded within a continuous matrix. The particles may be spherical or asymmetrical particles, as appreciated by those versed in the art of hydrogels.

In some examples, the polymeric matrix is in a form of a hydrogel holding, dispersed within the hydrogel, the first opioid antagonist in free form and liposomes encapsulating the second opioid antagonist.

In some examples, the pharmaceutical composition or the composite material is in dry form, e.g. lyophilized, such that when brought into contact with an aqueous medium, a hydrogel is formed, holding dispersed therein the liposomes encapsulating the second opioid antagonist and the first opioid antagonist in free form.

The polymeric matrix holds the liposomes.

The liposomes comprise at least one liposome forming lipid, which forms the liposomes' membrane. The liposomes' membrane is a bilayer membrane and may be prepared to include a variety of physiologically acceptable liposome forming lipids and, as further detailed below, non-liposome forming lipids (at the mole ratio which support the formation and maintenance of stable liposomes).

As used herein, the term “liposome forming lipids” is used to denote primarily glycerophospholipids and sphingomyelins which when dispersed in aqueous media by itself at a temperature above their solid ordered to liquid disordered phase transition temperature will form stable liposomes. The glycerophospholipids have a glycerol backbone wherein at least one, preferably two, of the hydroxyl groups at the head group is substituted by one or two of an acyl, alkyl or alkenyl chain, and the third hydroxyl group is substituted by a phosphate (phosphatidic acid) or a phospho-estar such as phopshocholine group (as exemplified in phosphatidylcholine), being the polar head group of the glycerophospholipid or combination of any of the above, and/or derivatives of same and may contain a chemically reactive group (such as an amine, acid, ester, aldehyde or alcohol). The sphingomyelins consists of a ceramide (N-acyl sphingosine) unit having a phosphocholine moiety attached to position 1 as the polar head group.

In the liposome forming lipids, which form the matrix of the liposome membrane the acyl chain(s) are typically between 14 to about 24 carbon atoms in length, and have varying degrees of unsaturation or being fully saturated being fully, partially or non-hydrogenated lipids. Further, the lipid matrix may be of natural source (e.g. naturally occurring phospholipids), semi-synthetic or fully synthetic lipid, as well as electrically neutral, negatively, or positively charged.

Examples of liposome forming glycerophospholipids include, without being limited thereto, glycerophospholipid. phosphatidylglycerols (PG) including dimyristoyl phosphatidylglycerol (DMPG); phosphatidylcholine (PC), including egg yolk phosphatidylcholine, dimyristoyl phosphatidylcholine (DMPC), 1-palmitoyl-2-oleoylphosphatidyl choline (POPC), hydrogenated soy phosphatidylcholine (HSPC), distearoylphosphatidylcholine (DSPC); phosphatidic acid (PA), phosphatidylinositol (PI), phosphatidylserine (PS).

The liposomes may also comprise other lipids (that do not form liposomes by themselves) typically used in the formation of liposomes, e.g. for stabilization, for affecting surface charge, membrane fluidity and/or assist in the loading of the active agents into the liposomes. Examples of such lipids can include sterols such as cholesterol (CHOL), cholesteryl hemisuccinate, cholesteryl sulfate, or any other derivatives of cholesterol.

The liposomes may further comprise lipopolymers. The term “lipopolymer” is used herein to denote a lipid substance modified by inclusion in its polar headgroup a hydrophilic polymer. The polymer headgroup of a lipopolymer is typically water-soluble. Typically, the hydrophilic polymer has a molecular weight equal or above 750 Da. Lipopolymers such as those that may be employed according to the present disclosure are known to be effective for forming long-circulating liposomes. There are numerous polymers which may be attached to lipids to form such lipopolymers, such as, without being limited thereto, polyethylene glycol (PEG), polysialic acid, polylactic (also termed polylactide), polyglycolic acid (also termed polyglycolide), apolylactic-polyglycolic acid, polyvinyl alcohol, polyvinylpyrrolidone, polymethoxazoline, polyethyloxazoline, polyhydroxyethyloxazoline, polyhydroxypropyloxazoline, polyaspartamide, polyhydroxypropyl methacrylamide, polymethacrylamide, polydimethylacrylamide, polyvinylmethylether, polyhydroxyethyl acrylate, derivatized celluloses such as hydroxymethylcellulose or hydroxyethylcellulose. The polymers may be employed as homopolymers or as block or random copolymers. The lipids derivatized into lipopolymers may be neutral, negatively charged, as well as positively charged. The most commonly used and commercially available lipids derivatized into lipopolymers are those based on phosphatidyl ethanolamine (PE), usually, distearoylphosphatidylethanolamine (DSPE).

One particular family of lipopolymers that may be employed according to the present disclosure are the monomethylated PEG attached to DSPE (with different lengths of PEG chains, in which the PEG polymer is linked to the lipid via a carbamate linkage resulting in a negatively charged lipopolymer, or the neutral methyl polyethyleneglycol distearoylglycerol (mPEG-DSG) and the neutral methyl poly ethyleneglycoloxy carbonyl-3-amino-1,2-propanediol distearoylester (mPEG-DS) [Garbuzenko O. et al., Langmuir. 21:2560-2568 (2005)]. Another lipopolymer is the phosphatidic acid PEG (PA-PEG).

The PEG moiety has a molecular weight of the head group is from about 750 Da to about 20,000 Da, at times, from about 750 Da to about 12,000 Da and typically between about 1,000 Da to about 5,000 Da. One specific PEG-DSPE commonly employed in liposomes is that wherein PEG has a molecular weight of 2000 Da, designated herein ²⁰⁰⁰PEG-DSPE or ^{2k}PEG-DSPE.

In general, the liposomes may have various shapes and sizes. In some examples, the liposomes employed in the present disclosure can be multilamellar vesicles (MLV) or multivesiclular vesicles (MVV).

MVV liposomes are known to have the form of numerous concentric or non-concentric, closely packed internal aqueous chambers separated by a network of lipid membranes and enclosed in a large lipid vesicle.

In some examples, the liposomes have a diameter that is at least 200 nm.

In some examples, the MVV are typically large multivesicular vesicles (LMVV), also known in the art by the term giant multivesicular vesicles (GMV). In accordance with one embodiment, the LMVV typically have a diameter in the range of about 200 nm and 25 μm, at times between about 250 nm and 25 μm.

In some other examples, the liposomes are small unilamellar vesicles, having a

The pharmaceutical composition and preferably the composite material disclosed herein are particularly suitable for intramuscular administration. Specifically, it has been realized that the composite material disclosed herein can be administered even by first responders with minimal training, e.g. via an auto-injector or any other suitable injector.

Thus, in accordance with another aspect disclosed herein there is provide a method of providing a prolonged counteraction against opioid overdose, the method comprises administration to a subject suffering from opioid overdose an amount of the disclosed pharmaceutical composition or composite material.

In some examples, the method comprises intramuscular administration of the said pharmaceutical composition or composite material.

In some further examples, the method comprises administration of the pharmaceutical composition once in every predetermined time intervals until plasma level of said opioid is non-detected or below a predetermined threshold.

As used herein, the forms “a”, “an” and “the” include singular as well as plural references unless the context clearly dictates otherwise. For example, the term “a liposome” includes one but also more liposomes within the pharmaceutical composition.

Further, as used herein, the term “comprising” is intended to mean that the composition of matter include the recited constituents, e.g. polymeric matrix, the first opioid antagonist, the second opioid antagonist, but not excluding other elements, such as physiologically acceptable carriers and excipients as well as other active ingredients. The term “consisting essentially of” is used to define composition which include the recited elements but exclude other elements that may have an essential significance on the effect to be achieved by the composition. “Consisting of” shall thus mean excluding more than trace amounts of other elements. Embodiments defined by each of these transition terms are within the scope of this disclosure.

Further, all numerical values, e.g. when referring the amounts or ranges of the elements constituting the composition comprising the elements recited, are approximations which are varied (+) or (−) by up to 20%, at times by up to 10% of from the stated values. It is to be understood, even if not always explicitly stated that all numerical designations are preceded by the term “about”.

The invention will now be exemplified in the following description of experiments that were carried out in accordance with the invention. It is to be understood that these examples are intended to be in the nature of illustration rather than of limitation. Obviously, many modifications and variations of these examples are possible in light of the above teaching. It is therefore, to be understood that within the scope of the appended claims, the invention may be practiced otherwise, in a myriad of possible ways than as specifically described hereinbelow.

### Non-Limiting Examples

### Materials

The materials used in the following formulations included:

| | |
|---|---|
| Naltrexone hydrochloride from - | Sigma, N3136, lot BCBX4989 |
| Naloxone hydrochloride dihydrate | Sigma, N7758, lot SLCB0098 |
| from - | |
| Ethanol abs from - | Merck, Emsure. Cat. 1.00983 |
| HSPC: Chol mix 3:1 weight ratio | Lipoid, lot no. 511740-2140002-01/ |
| from - | 001 |
| Monobasic sodium phosphate from - | Sigma, S8282, lot 046k0096 |
| Disodium phosphate dihydrate from - | Sigma, cat 30435, lot SZBA3290V |
| Ammonium sulfate from - | Merck, Emsure |
| Double distilled water (DDW) from- | In-house |

### Methods

### Naloxone and Naltrexone Concentration Assay

Naloxone and Naltrexone concentrations were determined using an HPLC assay previously described [M. Jafari-Nodoushan, J. Barzin, H. Mobedi, A stability-indicating HPLC method for simultaneous determination of morphine and naltrexone, J. Chromatogr. B Anal. Technol. Biomed. Life Sci. 1011 (2016) 163-170. doi:10.1016/j.jchromb.2015.12.048].

Specifically, the chromatographic conditions used include:

| | |
|---|---|
| Column | Luna 5 μm C18, 150 × 4.6 mm |
| Mobile phase | acetate buffer (10 mM, pH 4.0, containing 0.1% (w/w) |
| | 1-heptanesulfonic acid sodium salt) with acetonitrile |
| | in an 80:20 volumetric ratio |
| Flow rate | 1.5 ml/min |
| Detector | UV, 280 nm |
| Column | 30° C. |
| temperature | |
| Injection volume | 20 μl |

Lipid Concentration Assay
Lipid concentration was determined by HPLC using the following conditions:
Column & Packing: Phenomenex Jupiter C18, 5 μm 300 Å 150×4.6 mm.
Column Temperature: +40° C.
Autosampler +22° C. (thermostatic)
Temperature:
Mobile Phase: “A”—mix 500 ml of water and 500 ml of Methanol. Add 8 ml of TFA.
   “B”—mix 700 ml of 2-Propanol, 150 ml Methanol, 150 ml of THF. Add 8 ml of TFA.
Flow Rate: 1.0 ml/min
Detector: SofTA 300S ELSD
Detector Temperature: spray chamber: +40° C.
   drift tube: +80° C.
Gradient Program:

| Time (min) | % ″A″ | % ″B″ |
|---|---|---|
| 0 | 40 | 60 |
| 4 | 40 | 60 |
| 9 | 25 | 75 |
| 12 | 25 | 75 |
| 15 | 40 | 60 |
| 20 | 40 | 60 |

Injection Volume: 20 μl

### Liposome Preparation and Characterization

1A. Passive Loading of Both Naloxone and Naltrexone into MLV's

Naloxone, naltrexone and their combination (each referred to below as the drug solution) were solubilized in phosphate buffer pH 6.3. A mixture of HSPC and cholesterol (3:1 weight ratio) were mixed within a minimal amount of absolute ethanol and placed in a water bath at 65° C. until a clear solution was obtained. Drug solutions in phosphate buffer at 65° C. were added to the clear lipid solution in ethanol while stirring at 65° C. and left at 65° C. with stirring for 30 min. According to this method, the size of the liposomes is in the range of 0.2-20 μm.

Loading results are provided in Table 1.

TABLE 1
Passive loading of naloxone and naltrexone into MLV's liposomes

| | Liposomal | Liposomal | | |
|---|---|---|---|---|
| | naloxone | naltrexone | % | % |
| | conc. | conc. | Liposomal | Liposomal |
| | (mg/ml) | (mg/ml) | naloxone | naltrexone |
| Naloxone liposomes | 6.8 | | 24 | |
| Naltrexone liposomes | | 8.1 | | 22 |
| Naloxone + naltrexone | 12.2 | 13.0 | 38 | 36 |
| liposomes | | | | |

Naloxone and naltrexone loaded alone reached a liposomal concentration of 6.8 and 8.1 mg/ml respectively. Loading of both drugs to the same liposomes resulted in higher loading of 12.2 and 13.0 mg/ml, respectively.

1B. Remote Loading of Both Naloxone and Naltrexone into MLV's

MLV's containing ammonium sulfate 250 mM were prepared by hydrating HSPC: cholesterol (3:1 weight ratio) with ammonium sulfate. The extra-liposomal volume was washed three times in saline and reconstituted with sucrose 10% solution to result in MLV's having ammonium sulfate gradient. These MLV's were then incubated with naloxone, naltrexone and their combination at D/L molar ratio of 0.3 and 0.4. The size of the liposomes is assumed to be in the range of 1-25 μm.

Loading results are provided in Table 2.

TABLE 2
Remote loading of Naloxone, Naltrexone and their combination into
ammonium sulfate MLV's liposomes

| | Liposomal | Liposomal | | |
|---|---|---|---|---|
| | naloxone | naltrexone | % | % |
| | conc. | conc. | Liposomal | Liposomal |
| | (mg/ml) | (mg/ml) | naloxone | naltrexone |
| Naloxone liposomes | | | | |
| Molar D/L 0.3 | 2.2 | | 45 | |
| Molar D/L 0.4 | 2.5 | | 37 | |
| Naltrexone liposomes | | | | |
| Molar D/L 0.3 | | 2.8 | | 46 |
| Molar D/L 0.4 | | 3.3 | | 39 |
| Naloxone + naltrexone | | | | |
| liposomes | | | | |
| Molar D/L 0.3 | 1.7 | 2.1 | 38 | 39 |
| Molar D/L 0.4 | 1.5 | 1.9 | 26 | 28 |

Remote loading was similar to both drugs and ranged between 2.2-3.3 mg/ml liposomal drug concentration. The loading efficiency was higher than that obtained for the passive loading (37-46%, depending on the D/L ratio). Loading of both drugs to the same liposomes resulted in a slight decrease in loading (1.5-2.1 mg/ml) and loading efficiency (26-39%, depending on the D/L ratio).

1C. Remote Loading of Naloxone and Naltrexone into PEGylated Nano-Liposomes.

Naloxone and Naltrexone and their combination were remote loaded into PEGylated nano-liposomes (small unilamellear vesicles, SUV) having ammonium sulfate gradient. Loading results are provided in Table 3.

TABLE 3
Loading efficiency of naloxone and naltrexone into
PEGylated nano-liposomes

| | Liposomal | Liposomal | | |
|---|---|---|---|---|
| | Naloxone | Naltrexone | % | % |
| | conc. | conc. | Liposomal | Liposomal |
| | (mg/ml) | (mg/ml) | Naloxone | Naltrexone |
| Naloxone liposomes | | | | |
| Molar D/L 0.3 | 1.72 | | 49 | |
| Molar D/L 0.4 | 1.85 | | 35 | |
| Naltrexone liposomes | | | | |
| Molar D/L 0.3 | | 1.79 | | 48 |
| Molar D/L 0.4 | | 2.2 | | 40 |
| Naloxone + Naltrexone | | | | |
| liposomes | | | | |
| Molar D/L 0.3 | 0.67 | 0.94 | 27 | 29 |
| Molar D/L 0.4 | 0.85 | 0.95 | 23 | 22 |

Loading efficiency of the drugs remote loaded into nano-liposomes (size being are <100 nm) was similar to that obtained when loaded into large liposomes (MLV's, size being in the range of 1-10 μm).

Moreover, the decrease in loading efficiency when both drugs were loaded into the same liposomes was similar to that obtained for the large liposomes.

### 1D. Formulations Comprising Liposomal Naltrexone and Free Naloxone

Naltrexone was loaded into MLV's by either passive loading or remote/active loading. Specifically,

### Passive Loading

Lipid solution in ethanol was prepared by dissolving HSPC and cholesterol (3:1 weight ratio) in small volume of ethanol and incubating at 65° C. to achieve a clear solution. Naltrexone aqueous lipid hydration solution of 75 mg/ml was prepared in phosphate buffer 165 mM, pH 6.3 and heated to 65° C. The ethanolic lipid solution was added slowly to the aqueous phase at 65° C. while stirring for 30 min. HSPC concentration in this stage was ˜75 mg/ml. In cases of free naloxone in the formulation, the liposomes were centrifuged at 4° C. and the upper phase was replaced with 10 mg/ml naloxone solution in phosphate buffer 165 mM, pH 6.3.

### Remote/Active Loading

Lipid solution in ethanol was prepared by dissolving HSPC and cholesterol (3:1 weight ratio) in small volume of ethanol and incubating at 65° C. to achieve a clear solution. A solution of 250 mM ammonium sulfate was used as the aqueous hydration medium of the lipids. The ethanolic lipid solution was added slowly to the lipid hydration medium of 250 mM ammonium sulfate at 65° C. while stirring for 30 min. HSPC concentration in this stage was ˜75 mg/ml. The extra-liposomal ammonium sulfate was removed by three consecutive steps of centrifugation cycles at 4° C. and replacing the extraliposomal medium with 5% dextrose solution. The liposomes exhibiting trans-membrane ammonium gradient:

[(NH₄⁺)ₗᵢₚₒₛₒₘₑ>>[(NH₄⁺)_{medium}]

and high (250 mM) intra-liposome sulfate ions were then incubated with naltrexone solution at molar drug to lipid (D/L) ratio of 0.3-0.4 at 65° C. for 15 min.

In cases of free naloxone in the formulation, the liposomes were centrifuged at 4° C. and the upper phase was replaced with 10 mg/ml naloxone solution in phosphate buffer 165 mM, pH 6.3.

The formulation results are summarized in Table 4.

TABLE 4
Liposomal naltrexone/free naloxone formulations
prepared by passive and remote loading

| | Liposomal | Liposomal | Free | Free | HSPC | Naltrexone | Naltrexone | naloxone |
|---|---|---|---|---|---|---|---|---|
| Loading | naltrexone | naloxone | naltrexone | naloxone | concentration | to lipid | loading | sucked by |
| method | (mg/ml) | (mg/ml) | (mg/ml) | (mg/ml) | (mg/ml) | molar ratio | efficiency | liposomes |
| Passive | 1.37 | 0.17 | 3.74 | 5.35 | 53.05 | 0.05 | 2.6 | 3.1 |
| Remote | 2.68 | 1.38 | 1.80 | 4.69 | 45.52 | 0.12 | 29.4 | 22.7 |

A evident from Table 4, passive loading resulted in liposomal Naltrexone content of 1.37 mg/ml, which corresponds to 0.05 D/L molar ratio; this is considered a low yield formulation which is a direct result from the specific loading method and the a starting solution of only 75 mg/ml. This translate into a poor encapsulation efficiency of ˜ 2.6%.

Table 4 also shows that remote/active loading significantly higher loading efficiency of 29.4%, thus providing a higher liposomal concentration (2.68 mg/ml) and a D/L molar ratio of 0.12.

Free Naloxone was added externally to either types of liposomes (after the formation of naltrexone liposomes). It was found that the free Naloxone penetrate, to a small extent, into the passively loaded liposomes (0.17 mg/ml, 3.1% out of total naloxone concentration in the formulation) and to a higher extent, into the remote loaded liposomes (1.38 mg/ml corresponding to 22.7% of total naloxone concentration in the formulation). The higher penetration of naloxone into the remote loaded liposomes was somewhat expected as this antagonist is considered to be a weak base, thus was being affected by the ammonium sulfate gradient causing its loading into the liposomes.

### Drug Release

### Naltrexone Release

The release of naltrexone from liposomes was determined in the medium to which 25% sucrose solution and 25% serum were added to predict release in biological relevant media. In this medium the liposomes floated, which allowed to separate between precipitating free drug and drug encapsulated liposomes.

Table 5 summarizes the results.

TABLE 5
Release of naltrexone from passive and remote loaded liposomes

| Loading | % release naltrexone to | % increase in naloxone liposomal |
|---|---|---|
| method | the medium after 24 h | concentration after 24 h |
| Passive | 47 | 31 |
| Remote | 26 | 143 |

Table 5 shows that passively loaded naltrexone release from the liposomes was faster than that of naltrexone loaded into liposomes by remote/active loading. Specifically, after 24 h of incubation at 37° C., 47% of the passively loaded naltrexone was released as compared to only 26% release of naltrexone from the remote/active loaded liposomes. Concomitantly after 24 h incubation, naloxone was “pumped” from the medium into the liposomes having trans-membrane ammonium ion gradient, which was more significant as compared into the liposomes lacking such gradient (passive loading liposomes). In fact, passive loaded liposomes resulted with 0.25 mg/ml liposomal naloxone (31% intake) while for the remote loading liposomal naloxone reached 1.97 mg/ml (43% intake), showing again that naloxone was pumped into the liposomes by the trans-membrane ammonium ion the gradient.

### In Vitro/In Vivo Studies

The following combinations will be investigated:
Formulation containing liposomal Naltrexone and free Naloxone.
Formulation containing liposomal Naltrexone and free Naloxone and Doxapram.

The aim is to achieve high Naltrexone loading in the liposomes and slow in vitro and in vivo release for at least 72 h.

Different loading parameters are tested for their effect on the formulation performance, e.g. in terms of loading and drug release. These parameters will include active vs passive loading methods, incubation conditions, lipid composition and more.

The compatibility of Naloxone and Doxapram with liposomal Naltrexone will also be evaluated.

The obtained liposomes will be prepared in a hydrogel carrier.

The following assays will be used for formulation characterization:
In vitro release assay
Physical characterization: size, size distribution, medium pH, intra-liposome pH, conductivity, osmolality, trapped aqueous volume. Rate of drugs release, viscosity, injectability.
Pharmacokinetic: Selected formulations will be IM injected to mice. Plasma samples will be taken at different time points up to 1 week after administration. Target plasma concentrations for Naloxone will be 6-7 ng/ml at 10 min after administration. Naltrexone plasma target levels are >4 ng/ml for 48 h.
Bioanalytical LCMS/MS method will be developed prior to the in vivo study.
Optimization of the formulation: Based on the PK data, the correlation between the in vitro and in vivo PK data will be determined. The formulations will be optimized to achieve the PK exposure goals determined above. The optimization process will be based on the loading requirements and in vitro release assay (and its correlation to in vivo PK). Optimized formulations will be tested for their in vivo PK profile in mice.
Stability study of the optimized formulation will be performed. Samples will be placed at 4° C. and 15° C. stability chambers and will follow the formulation at several time points up to 2 years (for the 4° C. stability).

1-22. (canceled)

23. A pharmaceutical composition, comprising:
a first opioid antagonist; and
a second opioid antagonist;
wherein the first opioid antagonist has a half-life in plasma that is of shorter duration than a half-life of the second opioid antagonist in the plasma;
wherein the second opioid antagonist is encapsulated within liposomes.

24. The pharmaceutical composition of claim 23 wherein each of the first and second opioid antagonists is selected from the group consisting of naltrexone, naloxone, nalbuphine, butorphanol, pentazocine, diprenorphine and dihydroetorphine as well as opioid alkaloids and opioid peptides, and combinations thereof.

25. The pharmaceutical composition of claim 23, wherein the first opioid antagonist has a half-life in the plasma that is at least 20% shorter than the half-life of the second opioid antagonist.

26. The pharmaceutical composition of claim 23, wherein the first opioid antagonist is naloxone and the second opioid antagonist is naltrexone.

27. The pharmaceutical composition of claim 23, further comprising a respiratory stimulant.

28. The pharmaceutical composition of claim 27 wherein the respiratory stimulant is selected from the group consisting of 1-ethyl-4-(2-morpholin-4-ylethyl)-3,3-diphenyl-pyrrolidin-2-one (doxapram), and 4-amino-5-chloro-2-methoxy-N-(quinuclidin-3-yl)benzamide, and combinations thereof.

29. The pharmaceutical composition of claim 28 wherein the respiratory stimulant is doxapram.

30. A composite material, comprising:
a water insoluble, water absorbable polymeric matrix; and
a first opioid antagonist and a second opioid antagonist embedded or entrapped within the water insoluble, water absorbable polymeric matrix;
wherein the first opioid antagonist has a half-life in plasma that is shorter than a half-life of the second opioid antagonist in the plasma;
wherein the second opioid antagonist is encapsulated within liposomes.

31. The composite material of claim 30, wherein the each of the first and second opioid antagonists is selected from the group consisting of naltrexone, naloxone, nalbuphine, butorphanol, pentazocine, diprenorphine and dihydroetorphine as well as opioid alkaloids and opioid peptides, and combinations thereof.

32. The composite material of claim 30, wherein the first opioid antagonist has a half-life in the plasma that is at least 20%, shorter than the half-life of the second opioid antagonist.

33. The composite material of claim 30, wherein the first opioid antagonist is naloxone and the second opioid antagonist is naltrexone.

34. The composite material of claim 30, further comprising a respiratory stimulant.

35. The composite material of claim 34, wherein the respiratory stimulant is selected from the group consisting of 1-ethyl-4-(2-morpholin-4-ylethyl)-3,3-diphenyl-pyrrolidin-2-one (doxapram) and 4-amino-5-chloro-2-methoxy-N-(quinuclidin-3-yl)benzamide, and combinations thereof.

36. The composite material of claim 35, wherein the respiratory stimulant is doxapram.

37. A method of counteracting opioid overdose in a subject, the method comprising:
administering to the subject an amount of a pharmaceutical composition comprising a first opioid antagonist and a second opioid antagonist;
wherein the first opioid antagonist has a half-life in plasma that is shorter than a half-life of the second opioid antagonist in the plasma;
wherein the second opioid antagonist is encapsulated within liposomes.

38. The method of claim 37, wherein each of the first and second opioid antagonists is selected from the group comprising naltrexone, naloxone, nalbuphine, butorphanol, pentazocine, diprenorphine, dihydroetorphine, opioid alkaloids, opioid peptides, and combinations thereof.

39. The method of claim 37, wherein the first opioid antagonist has a half-life in the plasma that is at least 20%, shorter than the half-life of the second opioid antagonist.

40. The method of claim 37, wherein the first opioid antagonist is naloxone and the second opioid antagonist is naltrexone.

41. The method of claim 37, further comprising administering to the subject a respiratory stimulant.

42. The method of claim 37, wherein the first opioid antagonist and the second opioid antagonist are embedded or entrapped within a water insoluble, water absorbable polymeric matrix.
